SEQUENCE LISTING (3 sequences)

SEQ ID NO: 1 - protein, 365 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPAARATPQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 125 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGP

SEQ ID NO: 3 - protein, 240 aa, synthetic construct
AARATPQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0332216 A1
(43) Pub. Date: Oct. 30, 2025

(54) COMBINATION THERAPY FOR CLASSIC HODKIN'S LYMPHOMA

(71) Applicant: IMMUNEONCO BIOPHARMACEUTICALS (SHANGHAI) INC., Shanghai (CN)

(72) Inventors: Wenzhi TIAN, Shanghai (CN); Deqiang JING, Shanghai (CN); Qiying LU, Shanghai (CN); Zheng DONG, Shanghai (CN); Song LI, Shanghai (CN); Ruliang ZHANG, Shanghai (CN)

(21) Appl. No.: 18/647,455

(22) Filed: Apr. 26, 2024

(51) Int. Cl. A61K 38/17; A61K 9/00; A61K 39/00; A61P 35/00; C07K 16/28

(52) U.S. Cl. CPC A61K 38/1709; A61K 9/0019; A61P 35/00; C07K 16/2818; A61K 2039/505; A61K 2039/54; A61K 2039/545

(57) ABSTRACT

A method of treating classic Hodgkin's lymphoma (cHL) in a subject in need thereof, comprising intravenously administering to the subject i) a recombinant fusion protein at the dose of about 2.0 mg/kg body weight, once a week, and ii) an anti-PD-1 antibody at the dose of about 200 mg, once every 3 weeks, wherein the recombinant fusion protein comprises a mutated SIRPα D1 domain and a functional IgG1 heavy chain constant region, wherein the mutated SIRPα D1 domain comprises the amino acid sequence of SEQ ID NO: 2.

### INCORPORATION BY REFERENCE

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing XML labeled “55525-00084 SequenceListingXML” which was created on Mar. 22, 2024 and is 5 kb in size. The entire content of the sequence listing is incorporated herein by reference in its entirety.

### FIELD OF THE INVENTION

The disclosure relates to a pharmaceutical composition comprising a recombinant fusion protein and an anti-PD-1 antibody, wherein the recombinant fusion protein comprises a mutated SIRP-alpha D1 domain and a functional IgG1 heavy chain constant region. The disclosure also relates to a method for treating classic Hodgkin's lymphoma (cHL), especially relapsed or refractory cHL (R/R cHL), in a subject in need thereof using the pharmaceutical composition of the disclosure.

### BACKGROUND OF THE INVENTION

Lymphoma, as one of the most common malignant tumors, affects the lymphatic system which plays a critical role in both immune function and surplus extracellular fluid drainage. According to the report of the National Cancer Center in 2018, the lymphoma was the 10^{th} leading death cause in 2015, and its incidence was about 6.89 per 100,000 population. The lymphoma can be classified into Hodgkin's lymphoma (HL) and non-Hodgkin's lymphoma (NHL). The classic Hodgkin's lymphoma (cHL) accounts for about 90% of HL, and the 5-year total survival rate is 56% to 89% for advanced stage cHL patients (The guidelines for diagnosis and treatment of lymphoma (2018), by National Health Commission of the PRC). Currently the chemotherapies, including ABVD and AVD, achieve good efficacy in 75% to 85% cHL patients, with the overall response rate (ORR) above 90% (Lai C. et al., (2019) Blood and Lymphatic Cancer: Targets and Therapy 9:63-71; Song Y. et al., (2020) Leukemia 34(2): 533-542). However, some cHL patients are not responsive to such chemotherapies or suffer from the chemotherapy side effects. Approximately 10% to 15% of the early stage cHL patients and 15% to 30% late stage cHL patients develop relapsed or refractory (R/R) disease.

In recent years, the emergence of immunotherapies targeting e.g., PD-1/PD-L1 signaling holds promise for the treatment of the cHL patients. The anti-PD-1/PD-L1 antibodies, e.g., nivolumab, pembrolizumab, sintilimab, camrelizumab and tislelizumab, have been approved for clinical treatment of R/R cHL. In the phase II, open-label, single-arm, multicenter study (ClinicalTrials.gov Identifier NCT03209973), tislelizumab proved its efficacy and safety in treating R/R cHL as a single agent. Specially, 70 patients with relapsed or refractory cHL that failed to achieve a response or progressed after auto-stem cell transplant (SCT) or were ineligible for auto-SCT were enrolled, with 60 patients in the IIb-IV stage. These patients were administered with 200 mg tislelizumab every-3-weeks until progressive disease or unacceptable adverse side effects occurred, resulting in objective response rate (ORR) of 87.1% and complete remission rate (CR) of 67.1% with a median follow-up of 33.8 months. The 3-year progression-free survival (PFS) and overall survival rates were 40.8% and 84.8%, respectively (Song Y. et al., (2022) Clin Cancer Res. 28(6):1147-1156). The other anti-PD-1 antibodies, including pembrolizumab, nivolumab and sintilimab, achieved CR around 21% to 34% in R/R cHL treatment, meaning that most patients having received such monotherapies did not meet the remission criteria and need further treatment (Chen R. et al., (2019) Blood 134(14): 1144-1153). The combination of the anti-PD-1 and the chemotherapy may benefit those patients. For example, the combination of nivolumab and AVD achieved complete remission in 85% of early-stage unfavorable cHL patients. The complete remission rate was about 67% in the non-first line therapy using nivolumab and brentuximab vedotin, and camrelizumab in combination with decitabine achieved CR of 71%.

Magrolimab, a humanized IgG4 anti-CD47 antibody, is currently under the clinical trial for treating classic Hodgkin lymphoma in combination with pembrolizumab. Magrolimab, when binding to the CD47 molecules on tumor cells, may block CD47 binding to the signal regulatory protein alpha (SIRPα) on immune cells and thus enhance the ability of macrophages and other phagocytes to identify and destroy malignant cells. However, the FDA once placed a partial clinical hold on one Magrolimab clinical trial due to suspected unexpected serious adverse reactions.

The safety problem almost bothers every conventional anti-CD47 antibody, as in addition to tumor cells, normal human red blood cells also express CD47 proteins. The binding of the anti-CD47 antibodies to red blood cells may cause serious hemolysis and anemia. To mitigate the on-target anemia, a priming dose of the anti-CD47 antibodies may be administered to eliminate aging red cells selectively while sparing younger red cells, which lack prophagocytic signals.

There is always a desperate need for more effective therapies with less adverse side effects to treatment patients with classic Hodgkin lymphoma.

Citation or identification of any document in this application is not an admission that such document is available as prior art to the present invention.

### SUMMARY OF THE INVENTION

The inventors of the application designed a combination therapy for classic Hodgkin lymphoma (cHL) using a recombinant fusion protein and an anti-PD-1 antibody. The recombinant fusion protein comprises i) a mutated SIRPαD1, as a ligand trap for CD47 neutralization, and ii) a functional IgG1 heavy chain constant region such as the Fc region, which may induce recombinant fusion protein dependent cell mediated cytotoxicity or complement dependent cytotoxicity against cells bound by the mutated SIRPαD1. The mutated SIRPαD1 comprises a single asparagine to alanine point mutation at position 80 of the SIRPαD1, and shows higher binding capability to CD47s on Jurkat leukemia cells than the wild type SIRPαD1 and minimal binding to red blood cells.

According to the clinical trial data collected till February 2024, the combination therapy of the present application was well tolerated and achieved objective response rate (ORR) of 75% and disease control rate (DCR) of 100% in the cHL patients as enrolled. The combination therapy is especially potent in treating relapsed or refractory cHL (R/R cHL) patients that failed in the previous anti-PD-1 monotherapy. Further, the combination therapy of the present application does not require priming dose, and produced minimal hemolytic anemia.

Therefore, in a first aspect, the present disclosure provides a pharmaceutical composition that may comprise i) a recombinant fusion protein that may comprise a mutated SIRPαD1 and a functional IgG1 heavy chain constant region, and ii) anti-PD-1 antibody.

The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2.

The functional IgG1 heavy chain constant region may be an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region may be human IgG1 heavy chain constant region, or the Fc region thereof. The functional IgG1 heavy chain constant region may be the Fc region of human IgG1 heavy chain constant region comprising e.g., the amino acid sequence of SEQ ID NO: 3. The functional IgG1 heavy chain constant region may, in certain embodiments, consist of the amino acid sequence of SEQ ID NO: 3.

The recombinant fusion protein may comprise, from the N terminus to the C terminus, the mutated SIRPαD1 and the functional IgG1 heavy chain constant region. The recombinant fusion protein may comprise the amino acid sequence of SEQ ID NO: 1, and may consist of the amino acid sequence of SEQ ID NO: 1 in certain embodiments.

The anti-PD-1 antibody may be tislelizumab, nivolumab, pembrolizumab, sintilimab, or camrelizumab. In certain embodiments, the anti-PD-1 antibody may be tislelizumab.

The pharmaceutical composition of the disclosure may further comprise a pharmaceutically acceptable excipient.

In a second aspect, the disclosure may provide a method of treating classic Hodgkin's lymphoma (cHL) in a subject in need thereof, which may comprise intravenously administering to the subject i) a recombinant fusion protein at the dose of about 2.0 mg/kg body weight, once a week, and ii) an anti-PD-1 antibody at the dose of about 200 mg, once every 3 weeks.

The recombinant fusion protein may comprise a mutated SIRPαD1 and a functional IgG1 heavy chain constant region. The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2. The functional IgG1 heavy chain constant region may be an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region may be human IgG1 heavy chain constant region, or the Fc region thereof. The functional IgG1 heavy chain constant region may be the Fc region of human IgG1 heavy chain constant region comprising e.g., the amino acid sequence of SEQ ID NO: 3. The functional IgG1 heavy chain constant region may, in certain embodiments, consist of the amino acid sequence of SEQ ID NO: 3. The recombinant fusion protein may comprise, from the N terminus to the C terminus, the mutated SIRPαD1 and the functional IgG1 heavy chain constant region. The recombinant fusion protein may comprise the amino acid sequence of SEQ ID NO: 1, and may consist of the amino acid sequence of SEQ ID NO: 1 in certain embodiments.

The anti-PD-1 antibody may be tislelizumab, nivolumab, pembrolizumab, sintilimab, or camrelizumab. In certain embodiments, the anti-PD-1 antibody may be tislelizumab.

When the recombinant fusion protein and the anti-PD-1 antibody are administered on the same day, the recombinant fusion protein may be administered at least 30 minutes after completing the anti-PD-1 antibody administration.

The recombinant fusion protein of the disclosure may be administered via intravenous infusion. The intravenous infusion of the recombinant fusion protein may be done in 180±15 min for the first time, 120±15 min for the second time, and 60±15 min for subsequent infusions.

The anti-PD-1 antibody may be administered via intravenous infusion. The intravenous infusion of the anti-PD-1 antibody may be done in 60 min or more for the first time, and 30 min or more for the subsequence infusions if well tolerated.

In certain embodiments, the method may comprise the steps of:
(a) administering intravenously to the subject about 200 mg of the anti-PD-1 antibody in the form of a composition comprising a pharmaceutically acceptable excipient and the anti-PD-1 antibody,
(b) administering intravenously to the subject about 2.0 mg/kg body weight of the recombinant fusion protein in the form of a composition comprising a pharmaceutically acceptable excipient and the recombinant fusion protein, and
(c) after steps (a) and (b), repeating step (a) once every 3 weeks, and repeating step (b) once weekly.

The step (b) may be performed at least 30 minutes, e.g., about 30 minutes, after completing the administering of step (a).

Step (b) may be performed at least 30 minutes after completing step (a).

In step (c), the recombinant fusion protein may be administered about 30 minutes after completing the repeated administering of step (a) on the days that the subject is also having step (a) repeated.

The method may not include administering a priming dose of the recombinant fusion protein or dose ramp-up administrations of the recombinant fusion protein to mitigate on-target anemia.

The method may produce more than 60%, 65%, 70%, 75%, 80%, 85%, 90% or 95% of objective response rate after 24 weeks or longer of the treating. The objective response rate may be defined as the sum of complete response (CR) rate and partial response (PR) rate.

The method may produce more than 20%, 25%, 30%, 35%, 40%, or 45% of complete response rate after 24 weeks or longer of the treating.

The method may produce more than 85%, 90% or 95% of disease control rate after 24 weeks or longer of the treating. The disease control rate may be defined as the sum of complete response (CR) rate, partial response (PR) rate and stable disease (SD) rate.

In the method, no more than 5% or 10% of treated subjects have chance of treatment-related hemolytic anemia.

In the method, not more than 15% or 20% of treated subjects have chance of treatment discontinuation due to treatment-related adverse effects.

The subject may be human. The subject may be with relapsed or refractory cHL. The subject may have experienced prior anti-PD-1 treatment failure. In certain embodiments, the subject may have experienced prior tislelizumab treatment failure. In certain embodiments, the subject may be resistant to tislelizumab treatment. In certain embodiments, the subject may have experienced prior other non-tislelizumab PD-1 treatment failure. In certain embodiments, the subject may be resistant to other non-tislelizumab PD-1 antibodies.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### DESCRIPTION OF THE DRAWINGS

The following detailed description, given by way of example, but not intended to limit the invention solely to the specific embodiments described, may best be understood in conjunction with the accompanying drawings.

FIG. 1 shows the cHL patients' individual response and duration of response.

FIG. 2 shows the cHL patients' maximum percentage change from the baseline in tumor size.

### DETAILED DESCRIPTION OF THE INVENTION

The cancer cells may have developed several ways to escape from host immune surveillance. For example, the cancer cells may express on surfaces a high level of CD47 proteins which may bind to the signal regulatory protein alpha (SIRPα) on macrophage surfaces, thereby inducing inhibitory signals that inhibit the phagocytosis of cancer cells by macrophages. The tumor cells may also constitutively express PD-L1 molecules, which may bind to PD-1 molecules on immunes cells, causing T cell dysfunction and anergy and IL-10 secretion.

The signal regulatory protein (SIRP) is a trans-membrane glycoprotein, including three family members, SIRPα (CD172a), SIRPβ (CD172b) and SIRPγ (CD172g). All three proteins comprise similar extracellular regions but distinct intracellular domains. The extracellular region contains three immunoglobulin-like domains, one Ig V-set and two Ig C-set domains. The intracellular domain of SIRPα contains two inhibitory signaling regions that can inhibit signal transduction and corresponding cell functions. SIRPβ and SIRPγ have very short intracellular regions without any signal transduction domain. However, SIRPβ may function through an adaptor protein, e.g., DAP12 for signal transduction. SIRPs are mainly expressed on macrophages (Mφ), dendritic cells (DCs) and neurons.

CD47 is a transmembrane glycoprotein belonging to the immunoglobulin superfamily, and is expressed on the surface of all cell types including red blood cells. Ligands for CD47 include integrins, thrombospondin-1 and SIRPs. CD47, by interacting with SIRPα to emit a ‘don't eat me’ signal, can inhibit phagocytosis by macrophages and thus protects cells, such as blood cells, from being attacked by macrophages.

Studies have shown that many tumor or cancer cells over-express CD47s, which prevent phagocytosis of the cancer cells by macrophages. Cancer cells that over-express CD47 include cells of acute myeloid leukemia (AML), chronic myeloid leukemia (CML), acute lymphoblastic leukemia (ALL), non-Hodgkin's lymphoma (NHL), multiple myeloma (MM), bladder cancer, ovarian cancer, prostate cancer, lung cancer, colon cancer, breast cancer, and pancreatic cancer. It is reported that injection of CD47 specific antibody that blocks the binding of CD47 to SIRPα can significantly inhibit tumor growth in tumor-bearing mice. Tumor or cancer cells were eliminated completely when the same antibody was injected into the mice carrying human leukemia cells (Theocharides, A. P. A. et al., (2012) J. Exp. Med. 209(10): 1883-1899).

Conventional anti-CD47 antibodies bind intensely to CD47s on normal human red blood cell and thus produce an “antigen sink” effect, revealing comparatively less effect on tumor cells and requiring much higher dose (at 10-30 mg/kg) for efficacy. Additionally, to avoid hemolysis and anemia caused by CD47 binding on red blood cells, conventional anti-CD47 antibodies adopt an IgG4 or IgG2 design in their Fc region and may even require priming or “ramp-up” dose, as is the case for magrolimab. Consequentially, anti-CD47 antibodies reveal high hematological toxicity and minimal single-agent efficacy, and require combination therapy (with another tumor killing agent) to achieve in vivo anti-tumor efficacy (Joseph Maakaron et al., (2022) Blood 140 (Supplement 1): 3728-3730; David Andrew Sallman et al., (2023) Journal of Clinical Oncology 40(no. 16_suppl): 7017-7017; Naval Guastad Daver. Journal of Clinical Oncology 40(no. 16_suppl): 7020-7020).

In contrast, the mutated SIRPαD1-Fc molecule of present application utilizes an SIRPα extra cellular domain 1 as ligand trap for CD47 neutralization, which is more tumor-specific than anti-CD47 antibodies, and reveals minimal binding to red blood cell in vitro (data not shown). The SIRPαD1 of the mutated SIRPαD1-Fc molecule of present application contains a single asparagine to alanine point mutation at position 80 of the SIRPα sequence. As illustrated in FIG. 7B of U.S. Pat. No. 10,800,821B2, HY03M, an otherwise identical sequence having nine more amino acids at amino terminal than the mutated SIRPαD1-Fc molecule of present application, binds to CD47 proteins on Jurkat leukemia cells with higher affinity than the wild type SIRPαD1-Fc molecule does. Due to its high tumor specificity, the mutated SIRPαD1-Fc molecule of present application also adopts an IgG1 design in its Fc portion for significantly more effective single-agent anti-tumor efficacy. In fact, FIG. 10B of U.S. Pat. No. 10,800,821B2 shows that HY03MM, an IgG1 defective version of HY03M, has much less anti-tumor efficacy than the IgG1 competent HY03M version. Further, the mutated SIRPαD1-Fc molecule of present application requires no priming dose, and produces minimal hemolytic anemia and minimal grade 3 and 4 treatment-related hemolysis (Example 4).

PD-1 is an immune checkpoint molecule that is mainly expressed on memory T cells and exerts inhibitory effect on immune responses. PD-L1 and PD-L2 are PD-1's ligands. The PD-L1 molecules are constitutively expressed on antigen presenting cells, T cells, B cells, monocytes and epithelial cells, and their levels are upregulated in many cells in the presence of proinflammatory cytokines (Keir M E et al., (2008) supra; Chen J et al., (2016) Ann Oncol. 27(3):409-416). Studies have shown that the PD-L1 signaling may exert inhibitory effects on T cells in several ways, and also on B cell and NK cell-mediated lysis, and PD-1 or PD-L1 blockade by antibodies may lead to durable tumor regression in cancer patients (Dong H et al., (1999) Nat Med. 5(12):1365-1369; Keir M E et al., (2008) supra; Chen J et al., (2016) supra; Terme M et al., (2011) Cancer Res. 71(16):5393-5399; Fanoni D et al., (2011) Immunol Lett. 134(2):157-160). However, not all patients are responsive, and patients may develop relapsed or refractory (R/R) disease.

Classical Hodgkin's lymphoma (cHL) is a B-cell-derived malignancy characterized by a tumor microenvironment (TME) composed of a few malignant Hodgkin and Reed-Sternberg (HRS) cells, surrounded by leukocytes consisting of T cells, B cells, mast cells, macrophages, plasma cells, eosinophils and mesenchymal stromal cells. Studies have shown cHL patients with high CD47 expression on HRS cells had a significantly inferior event-free survival and overall survival (OS) (Gholiha A R et al., (2022) Br J Haematol. 197(5):580-589).

The present application provides a combination therapy for cHL patients using i) a recombinant fusion protein of the present application that binds CD47 and ii) an anti-PD-1 antibody. Such a therapy is well tolerated and particularly potent in treating relapsed or refractory cHL (R/R cHL) patients that failed in the previous anti-PD-1 monotherapy.

The recombinant fusion protein of the present application comprises i) a mutated SIRPαD1, as a ligand trap for CD47 neutralization, and ii) a functional IgG1 heavy chain constant region such as the Fc region, which may induce recombinant fusion protein dependent cell mediated cytotoxicity or complement dependent cytotoxicity against cells bound by the mutated SIRPαD1. An example of the recombinant fusion protein is the mutated SIRPαD1-Fc molecule comprising the amino acid sequence of SEQ ID NO: 1. Two copies of the recombinant fusion protein may dimerize to form an IgG1 antibody-like molecule through disulfide bonds between the functional IgG1 heavy chain constant regions. As an IgG1 antibody-like molecule, the recombinant fusion protein has the general structure of an IgG1 antibody, with the SIRPαD1 part being the “paratope” or CD47-binding portion.

The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2.

The “functional IgG1 heavy chain constant region” refers to an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region needs to contain the Fc region to exert said cytotoxicity.

The Fc region, i.e., the fragment crystallizable region, is the tail region of an antibody and is the domain that determines the effector function of the antibody, that is, how it engages with specific cell receptors or other defense proteins. An Fc region may interact with Fc receptors and/or proteins of the complement system, activating the immune system. For example, Fc receptors may bind to Fc-containing molecules (e.g., the antibodies, or the recombinant fusion proteins of the disclosure) that are attached to infected cells or invading pathogens, stimulating phagocytic or cytotoxic cells to destroy microbes or infected cells. Fc receptors (FcRs) are found on the surface of certain immune effector cells, including B lymphocytes, follicular dendritic cells, natural killer cells, macrophages, neutrophils, eosinophils, basophils, and mast cells.

The term “recombinant fusion protein dependent cell mediated cytotoxicity” herein refers to an immune mechanism through which the Fc receptor-bearing effector cells recognize and kill Fc-containing molecule (e.g., the recombinant fusion protein of the disclosure such as the one comprising the amino acid sequence of SEQ ID NO: 1)-coated target cells expressing tumor- or pathogen-derived antigens on their surface.

The “complement dependent cytotoxicity” is a mechanism by which a Fc-containing molecule (e.g., the recombinant fusion protein of the disclosure such as the one comprising the amino acid sequence of SEQ ID NO: 1) mediates specific target cell lysis through activation of an organism's complement system. CDC is initiated when C1q, the initiating component of the classical complement pathway, is fixed to the Fc portion of target cell-bound Fc-containing molecules.

In certain embodiments, the functional IgG1 heavy chain constant region may be the Fc region of human IgG1 heavy chain constant region comprising e.g., the amino acid sequence of SEQ ID NO: 3. The functional IgG1 heavy chain constant region may, in certain embodiments, consist of the amino acid sequence of SEQ ID NO: 3.

The recombinant fusion protein may comprise, from the N terminus to the C terminus, the mutated SIRPαD1 and the functional IgG1 heavy chain constant region. The recombinant fusion protein may comprise the amino acid sequence of SEQ ID NO: 1, and may consist of the amino acid sequence of SEQ ID NO: 1 in certain embodiments.

The anti-PD-1 antibody may be any anti-PD-1 antibody. In some embodiments, the anti-PD-1 antibody is tislelizumab.

The disclosure may provide a composition, e.g., a pharmaceutical composition, that may comprise the recombinant fusion protein and the anti-PD-1 antibody. The recombinant fusion protein may be formulated together with a pharmaceutically acceptable carrier. The anti-PD-1 antibody may be formulated together with a pharmaceutically acceptable carrier.

A pharmaceutically acceptable carrier means a carrier that is useful in preparing a pharmaceutical composition that is generally safe, non-toxic and neither biologically nor otherwise undesirable, and includes a carrier that is acceptable for veterinary use as well as human pharmaceutical use. The primary carrier used in a pharmaceutical composition may be either aqueous or non-aqueous in nature. For example, a suitable carrier may be water for injection, physiological saline solution or artificial cerebrospinal fluid, possibly supplemented with other materials common in injection. For example, the vehicle or carrier may be neutral buffered saline or saline mixed with serum albumin. Other exemplary pharmaceutical compositions comprise Tris buffers, or acetate buffers, which may further include sorbitol or a suitable substitute thereof. In one embodiment of the present disclosure, the compositions may be prepared for storage by mixing the selected recombinant fusion protein or anti-PD-1 antibody having the desired degree of purity with optional formulation agents in the form of a lyophilized cake or an aqueous solution. Further, the recombinant fusion protein and/or the anti-PD-1 antibody may be formulated as a lyophilizate using appropriate excipients such as sucrose, and may be reconstituted with the physiological saline (i.e., 0.9% sodium chloride solution) prior to administration.

The pharmaceutical composition can comprise any number of excipients. Excipients that can be used include carriers, surface active agents, thickening or emulsifying agents, binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003).

The recombinant fusion protein and the anti-PD-1 antibody may be formulated for intravenous, intramuscular, subcutaneous, parenteral, spinal or epidermal administration (e.g., by injection or infusion). Depending on the route of administration, the recombinant fusion protein or the anti-PD-1 antibody can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Particularly, the recombinant fusion protein and the anti-PD-1 antibody may be intravenously administered, e.g., by intravenous infusion.

The amount of the recombinant fusion protein or the anti-PD-1 antibody which can be combined with a carrier material to produce a single dosage form will vary depending upon the subject being treated, the particular mode of administration, the lesion site, etc. and will generally be that amount which produces a therapeutic effect.

Particularly, the recombinant fusion protein of the disclosure may be provided in the form of a protein dispersion, or lyophilized powder which can be dispersed in e.g., water for injection, whatever applicable.

The anti-PD-1 antibody of the disclosure may be provided in the form of a protein dispersion, or lyophilized powder which can be dispersed in e.g., water for injection, whatever applicable.

Dosage regimens may be adjusted to provide the optimum desired response (e.g., a therapeutic response). For example, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active molecule calculated to produce the desired therapeutic effect in association with the required pharmaceutical carrier. Alternatively, the recombinant fusion protein or the anti-PD-1 antibody can be administered as a sustained release formulation, in which case less frequent administration is required.

In another aspect, the present application provides a method for treating classic Hodgkin's lymphoma (cHL) in a subject in need thereof.

For phase Ib and phase II clinical studies, the inclusion criteria for patient cohorts include:
1. The subjects should be able to voluntarily sign the informed consent form and communicate well with the investigator, and should be willing to comply with study-related regulations;
2. Male or female ≥18 years of age;
3. Life expectancy ≥12 weeks;
4. clinical diagnosis:
   (1) Phase 1b: subjects diagnosed with advanced solid tumors by histology or cytology, who have failed in previous standard treatments (including anti-PD-1/PD-L1 monotherapy or combination therapy), or cannot receive standard treatments due to lack of such treatments for that particular tumor;
   (2) Phase 2: Subjects diagnosed histologically with cHL that experienced systemic second-line treatment failure or autologous stem cell transplant (ASCT) failure, including those a) that were responsive to the anti-PD-1 therapy but relapsed, or b) that were not responsive to the previous anti-PD-1 therapy.
5. The subjects should have at least one measurable tumor lesion according to RECIST v1.1 or 2014 Lugano Recommendations for Lymphoma (National Cancer Institute), one tumor lesion that received topical treatment such as radiation therapy but is progressing or worsening according to RECIST v1.1 can be deemed as measurable tumor lesion;
6. ECOG PS of 0 or 1;
7. Child-Pugh score of A or B without hepatic encephalopathy for a HCC patient;
8. Adequate organs function, including bone marrow, hepatic, renal, cardiac, coagulation;
9. Adverse events associated with previous anti-tumor therapy have returned to ≤grade 1 according to NCI CTCAE V5.0;
10. Upon signing the Informed Consent Form, females and males of childbearing potential must agree to practice effective contraception during the study and for 6 months after the last dose.

The exclusion criteria include:
1. Subjects who a) have received prior systemic anti-tumor therapy and are, prior to first dose of current trial, still within 4 weeks or 5 half-lives (T½) of the administration of the prior therapy, b) received hormone therapy, targeted therapy with small molecules, oral administration of fluorouracil-based drugs and/or endocrine therapy within 2 weeks prior to first dose of current trial, c) received topical palliative care within 2 weeks prior to first dose of current trial, d) received non-specific immunomodulatory therapy, excluding IL-11, within 2 weeks prior to first dose of current trial, e) took traditional Chinese medicine or proprietary Chinese medicine within 1 week prior to first dose of current trial;
2. Subjects that previously received treatment with monoclonal anti-CD47 antibodies or SIRPα-containing fusion proteins;
3. Subjects with symptomatic or progressive central nervous system (CNS) metastasis, excluding those a) who were cured of CNS metastasis with no new lesion within 2 weeks prior to first dose of current trial and stopped taking corticosteroids, if any, at least 3 days prior to first dose of current trial, b) who were not previously treated but with small CNS metastatic lesion(s) (length ≤1.5 cm);
4. Subjects with uncontrolled hypertension, pulmonary hypertension or unstable angina; myocardial infarction within 6 months prior to first dose of current trial; a history of chronic heart failure (NYHA G3/4); valvular disease; severe arrhythmia; CVA or TIA within 6 months prior to first dose of current trial;
5. Subjects with a history of arterial thrombosis, deep venous thrombosis or pulmonary embolism within 3 months prior to first dose of current trial;
6. Subjects that with a history of moderate or severe dyspnea; currently need oxygen inhalation therapy; or with interstitial lung disease (ILD) or severe pneumonia, severe chronic obstructive pulmonary disease, or severe pulmonary insufficiency;
7. Subjects with other malignant tumors within 5 years prior to first dose of current trial, excluding a) those cured of cervical carcinoma in situ or skin cancer (non-melanoma), b) those cured of a second primary tumor which was not relapsed within 5 years, c) with a second primary tumor that may benefit from the current trial;
8. Subjects with diseases that may cause gastrointestinal bleeding or perforation;
9. Subjects with uncontrollable pleural, peritoneal or pericardial effusions;
10. Subjects with ongoing hepatitis B (HBsAg positive and HBV-DNA level >predetermined lower limit) or hepatitis C (anti-HCV positive, and HCV RNA level >predetermined lower limit);
11. Subjects with a history of immunodeficiency, including HIV infection or organ transplantation;
12. Subjects with a history of autoimmune diseases and still need immunosuppression therapy or systemic hormone therapy, excluding those with hypothyroidism controllable by hormone replacement alone, skin diseases that do not need systemic treatment, controllable celiac disease;
13. Subjects with uncontrolled severe active infections within 4 weeks prior to first dose of current trial;
14. Subjects allergic to the study drug(s), chimeric or humanized antibodies or recombinant fusion proteins (>Grade 3 according to CTCAE5.0);
15. Subjects that took cancer treatment vaccines within 4 weeks prior to first dose of current trial or plan to take such vaccines;
16. Subjects who had major surgeries within 4 weeks prior to the first dose of current trial, or minor surgical procedures within 2 weeks prior to first dose of current trial, or those plan to have major surgeries within 1 week after taking the study drug(s);
17. Subjects with a history of neurological or psychiatric disorders, such as epilepsy and dementia, that will affect compliance;
18. Subjects with a history of alcohol abuse or drug abuse within 1 year;
19. Subjects who carry or breastfeed babies, or those who are not willing to practice effective contraception during the study and for 6 months after the last dose of current trial;
20. Subjects that experienced immunotherapy associated adverse events that caused permanent discontinuation;
21. Subjects with other situations rendering them inappropriate for participation in current clinical trial in the opinion of the investigator.

Subject screening should be done within 28 days after the subjects sign the informed consent form (ICF). The eligible subjects will be treated with the mutated SIRPαD1-Fc molecule of present application in combination with anti-PD-1.

During the treatment study, tumor assessment should be performed every 6 weeks±7 days after the first dose of study drug(s), and tumor imaging can be arranged for the subject(s) with suspected disease progression or new tumor lesion(s) or the subject(s) discontinuing the treatment for any reason. Subjects who finish the study should have tumor assessment every 12 weeks until disease progression, start of new anti-cancer treatment or death. The phase I and phase II trials should and to be ended upon occurrence of the last subject withdrawing consent, terminating treatment or withdrawing from the trial, lost to follow-up or death, being treated for 48 weeks or ending the study prematurely, whichever occurs first.

Subjects who have discontinued treatment for any reason are required to complete the safety follow-up visit (28±7 days after the last dose of study drug). Following completion of the safety follow-up period, these subjects will be followed for survival status. For subjects without disease progression or new anti-cancer treatment will continue to have tumor assessments (tumor imaging and assessment) every 12 weeks±7 days until end of the study, the start of new anti-cancer treatment, death, lost to follow-up or 12 months after last dose of study drug, whichever comes first. Once the start of new anti-cancer treatment, the follow-up is ended. For subjects that begin new treatment, the follow-up visit will be discontinued. The subjects with disease progression can end the follow-up visit. If subjects who can still benefit from study drug treatment if in the judgment of the investigator, treatment may continue after discussing with sponsor, but the subjects must be re-consented. Sponsor also reserves the right to terminate subjects' access to study drug(s) if any of the following occur (including but are not limited to): i) the sponsor terminates the study, ii) the subjects have other reasonable treatment options.

Pretreatments prior to the mutated SIRPαD1-Fc molecule infusion is recommended below in Table 1.

The clinical trial design for the treatment method of the disclosure is as follows.

The traditional 3+3 design is used for dose escalation and determination of dose-limiting toxicity (DLT), maximum tolerated dose (MTD) and recommended phase 2 dose (RP2D) of the mutated SIRPαD1-Fc molecule of present application+anti-PD-1 combination in phase I clinical trial.

According to the phase I clinical trial design, the mutated SIRPαD1-Fc molecule of present application is administrated via intravenous infusion at the dose of 1.0 mg/kg, 1.5 mg/kg or 2.0 mg/kg, once a week, 3 weeks as a treatment cycle, and the anti-PD-1 antibody is administered via intravenous infusion at a fixed dose of 200 mg, once every 3 weeks, 3 weeks as a treatment cycle. When the mutated SIRPαD1-Fc molecule of present application and anti-PD-1 administration is on the same day, the mutated SIRPαD1-Fc molecule infusion should be arranged at least 30 minutes after completing anti-PD-1 administration. The administration should be stopped for a subject when he/she experiences disease progression/death, intolerable toxicity, withdrawal of consent, loss to follow-up, 48 weeks of treatment, or discontinuation of treatment in the best interest of the patient in the opinion of the investigator, whichever occurs first.

TABLE 1
Pre-medications for infusion-related reactions

| Treatment | | |
|---|---|---|
| cycle | Timing | Pre-medications |
| First | 30~60 | Antipyretic analgesics, such as acetaminophen |
| pretreatment | mins | 0.5 g-0.65 g orally; indomethacin, 50 mg, orally |
| | before | or rectally. |
| | infusion | Antihistamines, such as diphenhydramine 50 mg |
| | | intramuscularly or intravenously; or |
| | | promethazine, 25 mg-50 mg, intramuscularly or |
| | | intravenously. |
| Second and | 30~60 | Antipyretic analgesics, such as acetaminophen |
| subsequent | mins | 0.5 g-0.65 g orally; indomethacin, 50 mg, orally |
| pretreatment | before | or rectally. |
| | infusion | Antihistamines, such as diphenhydramine 50 mg |
| | | intramuscularly or intravenously; or |
| | | promethazine, 25 mg-50 mg, intramuscularly or |
| | | intravenously. |
| | | Corticosteroids, such as dexamethasone 5 mg-10 |
| | | mg, intramuscularly or intravenously. |

Note:
1. The investigator should determine whether a subject that has received 3 pre-treatments should continue to take pre-treatments.
2. For subjects who developed infusion reactions after being pretreated with antihistamines and antipyretic analgesics, antihistamines, antipyretic analgesics or corticosteroids could be administered before subsequent infusions based on in the judgment of the investigator.

The primary objectives of phase I trial are i) to assess the Dose-limiting toxicities (DLTs) of the mutated SIRPαD1-Fc molecule of present application in combination with anti-PD-1 in subjects with advanced solid tumors, and ii) to determine the maximum tolerated dose (MTD) and recommended phase II dose (RP2D) of the mutated SIRPαD1-Fc molecule of present application in combination with anti-PD-1 in subjects with advanced solid tumors, including the type, incidence and severity of adverse events (AEs), changes in physical exam, laboratory tests and safety data (such as changes in physical examination results, laboratory tests, vital sign, ECG, ECHO according to CTCAE V5.0 criteria).

The secondary objectives are i) to assess the pharmacokinetics (PK) characteristics of the mutated SIRPαD1-Fc molecule of present application in combination with anti-PD-1 in subjects with advanced solid tumors, ii) to assess the immunogenicity of the mutated SIRPαD1-Fc molecule of present application in combination with anti-PD-1 in subjects with advanced solid tumors, iii) to evaluate the preliminary anti-tumor activity of the mutated SIRPαD1-Fc molecule of present application in combination with anti-PD-1 in subjects with advanced solid tumors. Tumor assessment should be done according to RECIST v1.1 and iRECIST, the parameters include objective response rate (ORR), duration of response (DoR), progression free survival (PFS), disease control rate (DCR) and time to response (TTR).

The exploratory objectives are i) to explore the relationship between drug exposure of the mutated SIRPαD1-Fc molecule of present application in combination with anti-PD-1 and efficacy & safety (if data allow), ii) to explore the correlation between predictive biomarkers and therapeutic indicators. The exploratory endpoints include Exposure-response (E-R) (the relationship between exposure of the mutated SIRPαD1-Fc molecule of present application in combination with anti-PD-1 and efficacy & safety), biomarkers (Relationship between PD-L1 expression and/or CD47 expression and response).

MTD is defined as the maximum dose at which the dose-limiting toxicity (DLT) is observed in 1/6 subjects or less. For the dose level to be determined as MTD, there must be at least 6 subjects with evaluable DLT data. Once MTD is determined, MTD is usually used as RP2D, or the dose level below MTD can be selected as RP2D.

RP2D was initially determined based on the safety, PK, efficacy, and other data collected in the current dose escalation study, in combination with the safety, tolerability and PK/PD data in the dose escalation study of the monotherapy of the mutated SIRPαD1-Fc molecule of present application for treating lymphoma (which is an unpublished study), and finally decided by the scientist review committee (SRC). The RP2D was also approved by Center for Drug Evaluation for phase II trial in cHL treatment.

For phase II clinical study, cHL patients having been diagnosed histologically with cHL and experienced systemic second-line treatment failure or autologous stem cell transplant (ASCT) failure were and are being enrolled. In phase II study, the mutated SIRPαD1-Fc molecule of present application is administrated via intravenous infusion at the dose of 2.0 mg/kg, once a week, 3 weeks as a treatment cycle, and the anti-PD-1 antibody is administered via intravenous infusion at a fixed dose of 200 mg, once every 3 weeks, 3 weeks as a treatment cycle. When the mutated SIRPαD1-Fc molecule of present application and anti-PD-1 administration is on the same day, infusion of the mutated SIRPαD1-Fc molecule of present application should be arranged at least 30 minutes after completing anti-PD-1 administration.

The primary objective of the phase II trial is to evaluate the preliminary anti-tumor activity of the mutated SIRPαD1-Fc molecule of present application in combination with anti-PD-1 in subjects with lymphoma. The primary endpoints include the ORR according to RECIST v1.1 or Luagano 2014.

The secondary objectives are i) to assess the safety, tolerability of the mutated SIRPαD1-Fc molecule of present application in combination with anti-PD-1 in subjects with lymphoma, ii) to assess the immunogenicity of the mutated SIRPαD1-Fc molecule of present application in combination with anti-PD-1 in subjects with lymphoma, iii) to evaluate the preliminary anti-tumor activity of the mutated SIRPαD1-Fc molecule of present application in combination with anti-PD-1 in subjects with lymphoma. The secondary endpoints include i) type, incidence and severity of adverse events (AEs), changes in physical exam, laboratory tests and safety data (such as changes in physical examination results, laboratory tests, vital sign, ECG, ECHO according to CTCAE V5.0 criteria), ii) positive rates of anti-drug antibodies (ADA) and neutralizing antibodies (NAb), iii) ORR according to iRECIST; DoR, PFS, DCR and TTR according to RECIST v1.1, iRECIST or Lugano 2014.

The exploratory objectives are i) to explore the population pharmacokinetics (Pop-PK) characteristics of the mutated SIRPαD1-Fc molecule of present application in lymphoma (if data allow), ii) to explore the correlation between exposure of the mutated SIRPαD1-Fc molecule of present application and efficacy and safety (if data allow), iii) to explore the correlation between predictive biomarkers and therapeutic indicators. The exploratory endpoints include Pop-PK (PK pharmacokinetics of the mutated SIRPαD1-Fc molecule of present application in lymphoma), E-R (relationship between exposure of the mutated SIRPαD1-Fc molecule of present application and efficacy and safety endpoints), Biomarker (relationship between PD-L1 expression and/or CD47 expression and response).

Objective response rate (ORR) is a measure of how a specific treatment impacts tumor burden in a patient with a history of tumors, and is defined as the proportion of patients that respond either partially or fully to therapy.

Disease control rate (DCR) describes the percentage of patients with advanced cancer whose therapeutic intervention has led to a complete response, partial response, or stable disease.

Complete response or complete remission (CR) is usually defined as the lack of detectable evidence of tumor. Partial response or partial remission (PR) implies that further treatment will probably be required to attempt a cure. In some slow-growing tumors (including low-grade lymphomas) no further treatment may be immediately necessary until the disease starts increasing in size again. Stable disease (SD) is usually used to describe a tumor that is neither growing nor shrinking. Progressive disease (PD) is a term that describes a disease that is progressing or worsening.

During the trial, palliative and supportive treatments for symptoms associated with advanced tumors are allowed, and drugs for adverse reactions are also permitted. Drugs that may induce or aggravate clinical study symptoms cannot be used, and symptom monitoring should be strengthened if medication of such drugs cannot be avoided.

Palliative local radiotherapy (within limited range) for pain relief is permitted, and bisphosphonates or denosumab are permitted if bone metastases have already occurred before enrollment. Subjects can use topical, ocular, intra-articular, intranasal, and inhaled corticosteroids. Systemic corticosteroids at physiologic replacement doses (i.e., prednisone ≤10 mg/day) are permitted. Short-term use of corticosteroids for prophylaxis (e.g., contrast media allergy), for non-autoimmune disorders (e.g., delayed hypersensitivity reactions due to contact allergens), or for management of adverse reactions caused by the investigational drug is permitted.

Oral contraceptives, hormone replacement therapy, prophylactic or therapeutic anticoagulants, and other permitted therapies may be continued.

The following treatments other than the investigational drugs are prohibited during the study:
1. Any concomitant anti-tumor treatments (chemotherapy, immunotherapy, biologic product, extensive radiation therapy, hormonal therapy, targeted therapy, surgery, interventional therapy, and device therapy), investigational therapy, or approved therapy are not allowed;
2. Immunosuppressants are not allowed, except for such drugs are used to treat immune-related adverse events;
3. Systemic corticosteroids with immunosuppressive effects are not allowed, except for temporary hormone replacement therapy for infusion reactions, immune-related adverse reactions, or adrenal insufficiency, in such cases, dosage of systemic corticosteroids for infusion reactions or immune-related adverse reactions shall be controlled at the daily dose of ≤10 mg prednisone or equivalent, and shall be reduced gradually before next dose. The administration of the mutated SIRPαD1-Fc molecule of present application should be temporarily discontinued if >10 mg prednisone is taken daily;
4. Any Traditional Chinese Medicine approved for anti-cancer therapy is not allowed;
5. Immunization of live or attenuated vaccine is not allowed throughout the study.

The sample sizes in statistical analysis are described as follows.

The phase Ib study uses a 3+3 approach for dose escalation, with 3 to 6 patients in each dose group. The phase II cohort plans to enroll more than 15 subjects.

Descriptive statistics are used for safety data. The dose-limiting toxicities (DLT) occurred in each dose group are studied in phase Ib trial and all adverse events (AEs), treatment related adverse events (TRAEs), AEs causing death, AEs causing dose discontinuation and the like are analyzed according to the severity (NCI-CTCAE 5.0), system organ class (SOC) and/or preferred term (PT).

The numbers of subjects with complete remission (CR), partial remission (PR), stable disease (SD), and progressive disease (PD) are calculated according to RECIST v1.1 or Lugano 2014, objective response rate (ORR, CR+RR) and disease control rate (DCR, CR+PR+SD) are determined based on subjects' best overall response. The time to event (time to CR, PR, SD or PD), DCR, time to response (TTR), and progression free survival (PFS) are analyzed using Kaplan-Meier.

The numbers of subjects with complete remission (iCR), partial remission (iPR), stable disease (iSD), unconfirmed progressive disease (iUPD), confirmed progressive disease (iCPD) according to iRECIST are calculated, objective response rate (iORR, iCR+iRR) and disease control rate (iDCR, iCR+iPR+iSD) are determined based on subjects' best overall response. The time to event (time to CR, PR, SD or PD), iDCR, time to response (iTTR), and progression free survival (iPFS) are analyzed using Kaplan-Meier.

The clinical trial information of the current combination therapy, including the drug name, the dose levels and the condition or disease to be treated, was published on ClinicalTrials.gov with the identifier NCT05833984 on Apr. 27, 2023. The subject matter disclosed on Clinicaltrials.gov was the disclosure of the inventors' and applicant's own information and as such the Clinicaltrials.gov disclosure is not to be considered as, and is excepted from being, prior art as to the inventors and applicant.

According to the data as collected by now, the mutated SIRPαD1-Fc molecule of present application does not cause obvious hemagglutination, and thus the administration of the mutated SIRPαD1-Fc molecule of present application requires no priming dose or dose ramp-up administrations, making the combination therapy of the disclosure more safe and more convenient. Further, the mutated SIRPαD1-Fc molecule of present application has a high binding affinity to the CD47-expressing tumor cells, no more than 2.0 mg/kg of the mutated SIRPαD1-Fc molecule of present application can produce single agent in vivo efficacy.

Most importantly, the mutated SIRPαD1-Fc molecule of present application +anti-PD-1 combination achieved ORR and DCR of 66.7% and 93.9%, respectively, in cHL patients, suggesting the combination therapy of the disclosure may benefit most cHL patients. As the cHL patients enrolled in Phase II trial all experienced anti-PD-1 treatment failure, the current combination therapy of the disclosure may be a good choice for such a patient cohort. Overall, the combination therapy was well tolerated. Fourteen (14) out of 33 patients had clinically insignificant G3/4 TRAEs and the most common G3/4 TRAE was transient lymphocyte count decrease. The mutated SIRPαD1-Fc molecule associated lymphopenia is transient, occurred immediately after the mutated SIRPαD1-Fc molecule infusion and recovered back to bassline within a few hours to 2-3 days. It is believed this phenomenon was caused by lymphocyte redistribution and lymphocytes were not damaged. And the mutated SIRPαD1-Fc molecule of present application does not cause myelosuppression in bone marrow. Decrease of platelet and hemoglobin was transient and generally returned to baseline in 4 hours to 8 days. No treatment related hemolytic anemia and no permanent discontinuation of study occurred.

The present disclosure is now further described with the non-limiting examples below.

### EXAMPLES

### Example 1. Phase Ib Dose Escalation Study of Mutated SIRPαD1-Fc+Tislelizumab Combination

The traditional 3+3 design was used for dose escalation and determination of dose-limiting toxicity (DLT), maximum tolerated dose (MTD) and recommended phase 2 dose (RP2D) of the combination of the mutated SIRPαD1-Fc molecule of present application comprising the amino acid sequence of SEQ ID NO: 1 and azacutudube (AZA) for cHL treatment.

Briefly, according to the phase I clinical trial design, the subjects that had advanced solid tumors and passed the screening process were administered with i) tislelizumab (manufactured by BeiGene) via intravenous infusion at a fixed dose of 200 mg, once every 3 weeks, 3 weeks as a treatment cycle, and ii) the mutated SIRPαD1-Fc molecule of present application comprising the amino acid sequence of SEQ ID NO: 1 in 0.9% physiological saline solution via intravenous infusion at the dose of 1.0 mg/kg, 1.5 mg/kg or 2.0 mg/kg, once a week, 3 weeks as a treatment cycle. The tislelizumab infusion was done in 60 min or more for the first time, and 30 min or more for the subsequent infusions if well tolerated. The infusion of the mutated SIRPαD1-Fc molecule was done in 180±15 min for the first time, 120±15 min for the second time, and 60±15 min for subsequent infusions. When the mutated SIRPαD1-Fc molecule and tislelizumab administration was on the same day, the infusion of the mutated SIRPαD1-Fc molecule was arranged at least 30 minutes after completing tislelizumab administration. The administration was to stop when a patient experienced disease progression/death, intolerable toxicity, withdrawal of consent, loss to follow-up, 48 weeks of treatment, or discontinuation of treatment in the best interest of the patient in the opinion of the investigator, whichever occurred first.

The subjects were monitored for the occurrence of adverse events (AEs, including type, incidence, duration and severity), and their physical status (e.g., the test results of ECG and ECHO), based on Common Terminology Criteria for Adverse Events (CTCAE) v5.0. The pharmacokinetics (PK) characteristics, immunogenicity and preliminary anti-tumor activity of the mutated SIRPαD1-Fc+tislelizumab combination were also measured.

Tumor assessment was performed every 6 weeks±7 days after the first dose of study drug(s), and tumor imaging can be arranged for the subject(s) with suspected disease progression or new tumor lesion(s) or the subject(s) discontinuing the treatment for any reason. Subjects who finished the study should have tumor assessment every 12 weeks until disease progression, start of new anti-cancer treatment or death.

The phase I trial was ended upon occurrence of the last subject withdrawing consent, terminating treatment or withdrawing from the trial, lost to follow-up or death, being treated for 48 weeks or ending the study prematurely, whichever occurred first.

The DLT refers to toxicity, including the hematologic toxicity, non-hematologic toxicity, infusion-related reactions and other toxicity that causes death(s) unattributable to tumor progression, observed within 1 to 21 days after the first dose that may be related to the study drug(s). Hematologic toxicity includes: 1) Grade 4 neutropenia lasting for more than 5 days, 2) ≥Grade 3 neutropenia with fever (neutrophil count less than 1.0×10⁹/L, body temperature higher than 38.3° C. or body temperature higher than 38.0° C. for more than 1 hour), 3) thrombocytopenia (≥Grade 3 thrombocytopenia with bleeding, or in need of platelet infusion; Grade 4 thrombocytopenia without symptoms, with platelet count less than 25×10⁹/L for more than 24 hours), 4) ≥Grade 3 anemia, 5) Grade 4 anemia, and 6) other Grade 4 hematologic toxicity events. Non-hematologic toxicity includes: 1) ≥Grade 3 toxicity to important organs, including the heart, lung(s), gastrointestinal tract, liver(s), kidney(s) and the neural system, 2) other ≥Grade 4 non-hematological toxicity events, except for a. Grade 3 rash, nausea/vomiting, diarrhea and/or electrolyte imbalance that can be alleviated to ≤Grade 2 within 72 hours post best supportive treatment, b. Grade 3 fatigue lasting ≤7 days, c. ≤Grade 3 endocrine system disease that can be controlled by hormone replacement therapy, d. Grade 3 tumor lysis syndrome or related electrolyte imbalance that can be alleviated to ≤Grade 2 within 7 days, e. Grade 3 alanine aminotransferase (ALT), aspartate aminotransferase (AST), and/or alkaline phosphatase (ALP) level increase that can be alleviated to ≤Grade 2 within 7 days with relative treatment, and 3) Grade 3 cytokine release syndrome (CRS) that cannot be alleviated to Grade 1 or the baseline within 7 days. The infusion-related reactions/allergic reactions (IRRs) are generally adverse reactions that often occur during intravenous infusion of protein drugs, and are not considered DLTs except for Grade 4 IRR. When Grade 4 IRR was observed, the treatment to that subject was to be disconnected and the subject should be treated. If more than 2 subjects experience Grade 4 IRR, the enrollment should be suspended, and the sponsor management committee (SMC) should discuss whether to stop enrollment.

MTD is defined as the maximum dose at which the dose-limiting toxicity (DLT) is observed in 1/6 subjects or less. For the dose level to be determined as MTD, there must be at least 6 subjects with evaluable DLT data. Once MTD is determined, MTD is usually used as RP2D, or the dose level below MTD can be selected as RP2D.

RP2D was initially determined based on the safety, PK, efficacy, and other data collected in the current dose escalation study, in combination with the safety, tolerability and PK/PD data in the dose escalation study of the monotherapy of the mutated SIRPαD1-Fc molecule of present application for treating lymphoma (which is an unpublished study), and finally decided by the scientist review committee (SRC). The dosing regimen of the mutated SIRPαD1-Fc molecule at 2.0 mg/kg was finally chosen as the recommended phase 2 dose (RP2D) for cHL patients, especially R/R cHL patients.

### Example 2. Phase II Clinical Trial of Mutated SIRPαD1-Fc Molecule+Tislelizumab Combination in cHL Patients

In the phase II clinical trial, cHL patients were and are being enrolled according to the inclusion and exclusion criteria described in the DETAILED DESCRIPTION OF THE INVENTION part above. Basically, the cHL patients having been diagnosed histologically with cHL and experienced systemic second-line treatment failure or autologous stem cell transplant (ASCT) failure were and are being enrolled.

The cHL patients, once enrolled, were administered with i) tislelizumab via intravenous infusion at a fixed dose of 200 mg, once every 3 weeks, 3 weeks as a treatment cycle, and ii) the mutated SIRPαD1-Fc molecule of present application comprising the amino acid sequence of SEQ ID NO: 1 via intravenous infusion at the dose of 2.0 mg/kg, once a week, 3 weeks as a treatment cycle. The tislelizumab infusion was done in 60 min or more for the first time, and 30 min or more for the subsequence infusions if well tolerated. The infusion of the mutated SIRPαD1-Fc molecule was done in 180±15 min for the first time, 120±15 min for the second time, and 60±15 min for subsequent infusions. When the mutated SIRPαD1-Fc molecule and tislelizumab were to be administered on the same day, the infusion of the mutated SIRPαD1-Fc molecule was arranged at least 30 minutes after completing tislelizumab administration.

Tumor assessment was performed every 6 weeks±7 days after the first dose of study drug(s), and tumor imaging can be arranged for the subject(s) with suspected disease progression or new tumor lesion(s) or the subject(s) discontinuing the treatment for any reason. Subjects who finished the study should have tumor assessment every 12 weeks until disease progression, start of new anti-cancer treatment or death.

The administration was to stop when a patient experienced disease progression/death, intolerable toxicity, withdrawal of consent, loss to follow-up, 48 weeks of treatment, or discontinuation of treatment in the best interest of the patient in the opinion of the investigator, whichever occurred first.

### Example 3. Efficacy of Mutated SIRPαD1-Fc Molecule Together with Tislelizumab in R/R cHL Patients

As of Feb. 19, 2024, 33 patients with R/R cHL were enrolled and treated with the mutated SIRPαD1-Fc molecule of present application+tislelizumab combination. All these patients previously received at least one regimen containing anti-PD-1, 14 of which were resistant to prior tislelizumab treatment, and 21 of which were resistant to prior other non-tislelizumab PD-1 antibody treatment.

The baseline characteristics of these patients were summarized in Table 2.

TABLE 2
Baseline characteristics of the cHL patients

| Characteristics | N = 33 |
|---|---|
| Age, years | |
| Median (range) | 35 (19-77) |
| Gender, n (%) | |
| Male | 23 (69.7) |
| Female | 10 (30.3) |
| ECOG PS, n (%) | |
| 0 | 22 (66.7) |
| 1 | 11 (33.3) |
| Ann Arbor Staging, n (%) | |
| II | 1 (3.0) |
| III | 10 (30.3) |
| IV | 22 (66.7) |
| Bulky (>=10 cm), n (%) | |
| Yes | 2 (6.7) |
| No | 30 (90.9) |
| unknown | 1 (3.0) |
| Prior systemic anti-cancer therapy, n (%) | |
| Median (range) | 4 (2-15) |
| second line (2 L) | 5 (15.2) |
| 3 L | 10 (30.3) |
| 4 L | 4 (12.1) |
| 5 L | 5 (15.2) |
| >5 L | 9 (27.3) |

Table 3 summarized the efficacy data in phase II as recorded as of Feb. 19, 2024. Among the 33 response evaluable patients, 8, 14 and 9 achieved complete remission (CR), partial remission (PR), and stable disease (SD), respectively. The objective response rate (ORR) was 66.7% and disease control rate (DCR) was 93.9%.

The time to response (TTR) was about 2.1 months.

TABLE 3
Efficacy of the mutated SIRPαD1-Fc molecule of present
application + tislelizumab combination in R/R cHL patients

| | Efficacy Evaluable |
|---|---|
| Best Response | (N = 33) |
| CR, n (%) | 8 (24.2) |
| PR, n (%) | 14 (42.4) |
| SD, n (%) | 9 (27.3) |
| PD, n (%) | 2 (6.1) |
| ORR, n (%) | 22 (66.7) |
| DCR, n (%) | 31 (93.9) |

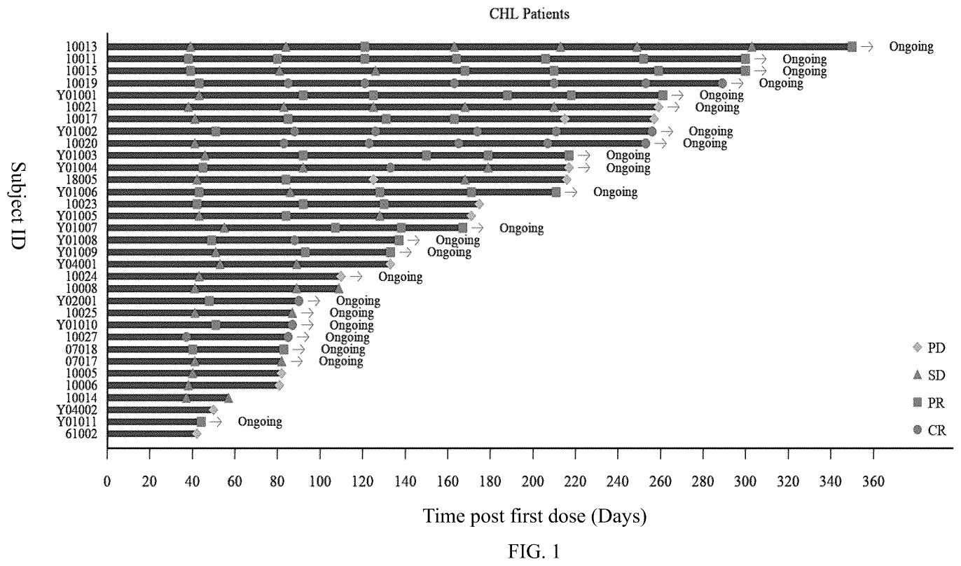

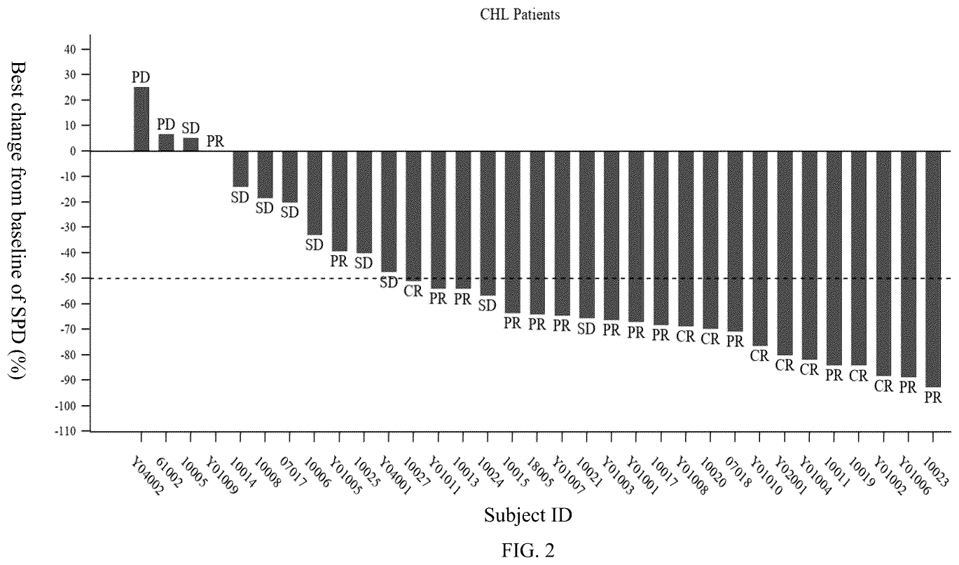

The patients' individual response and duration of response (DOR) was shown in FIG. 1. Most of the patients achieved SD at around Day 30 to 40 and then PR or CR around Day 80 post first dose of study drug(s), and a few patients achieved PR around Day 30 to 40. The patients' maximum percentage change from the baseline, calculated as the lesion SPD (sum of products of diameters), was shown in FIG. 2. More than half of these patients had a >50% reduction in SPD.

Among the 14 tislelizumab resistant patients (see Table 4), 2 achieved CR, 9 achieved PR, and 3 achieved SD. ORR was 78.6% and DCR was 100%. Among the 21 patients resistant to other non-tislelizumab anti-PD-1 antibodies (Table 4), 6 achieved CR, 6 achieved PR, and 7 achieved SD. ORR was 57.1% and DCR was 90.5%. This suggested that the mutated SIRPαD1-Fc molecule+tislelizumab combination might have potent efficacy in treatment of the R/R cHL patients that failed in the previous anti-PD-1 monotherapy.

TABLE 4
Efficacy of the mutated SIRPαD1-Fc molecule of present
application + tislelizumab combination in tislelizumab or
non-tislelizumab anti-PD-1 resistant cHL patients

| | Tislelizumab resistant | Other anti-PD-1 resistant |
|---|---|---|
| | patients (N = 14) | patients (N = 21) |
| CR, n (%) | 2 (14.3) | 6 (28.6) |
| PR, n (%) | 9 (64.3) | 6 (28.6) |
| SD, n (%) | 3 (21.4) | 7 (33.3) |
| PD, n (%) | 0 | 2 (9.5) |
| ORR, n (%) | 11 (78.6) | 12 (57.1) |
| DCR, n (%) | 14 (100) | 19 (90.5) |

### Example 4. Safety of Mutated SIRPαD1-Fc Molecule Together with Tislelizumab in R/R cHL Patients

The treatment related adverse events (TRAEs) observed during the treatment of the mutated SIRPαD1-Fc molecule of present application+tislelizumab combination were summarized in Table 5.

TABLE 5
Treatment related adverse events in the mutated SIRPαD1-Fc
molecule of present application + tislelizumab combination therapy

| | Patients (n = 33) | |
|---|---|---|
| Patients, n (%) | All Grades | G3-4 |
| TRAEs | 33 (100) | 14 (42.4) |
| Platelet count decrease | 14 (42.4) | 4 (12.1) |
| Anemia | 11 (33.3) | 1 (3.0) |
| Anti-erythrocyte antibody positive | 4 (12.1) | 0 |
| Prothrombin level increase | 4 (12.1) | 0 |
| White blood cell count decrease | 16 (48.5) | 4 (12.1) |
| Lymphocyte count decrease | 10 (30.3) | 10 (30.3) |
| Neutrophil count decrease | 11 (33.3) | 4 (12.1) |
| Infusion related reaction | 5 (15.2) | 0 |
| Hepatic function abnormal | 6 (18.2) | 1 (3.0) |
| Gamma-glutamyl transferase increase | 4 (12.1) | 0 |
| Blood bilirubin increase | 4 (12.1) | 0 |
| Electrocardiogram ST-T change | 5 (15.2) | 0 |
| Fatigue | 4 (12.1) | 0 |

Generally speaking, the mutated SIRPαD1-Fc molecule plus tislelizumab combination was well tolerated. The most common TRAEs (≥20%) were white blood cell count decrease (48.5%), platelet count decrease (42.4%), anemia (33.3%), lymphocyte count decrease (30.3%), and neutrophil count decrease (33.3%). Fourteen (14) patients out of 33 (42.4%) had G3/4 TRAEs, the most common G3/4 TRAE (≥20%) was lymphocyte count decrease.

Overall, the mutated SIRPαD1-Fc molecule associated lymphopenia is transient, occurred immediately after the infusion of the mutated SIRPαD1-Fc molecule and recovered back to bassline within a few hours to 2-3 days. It is believed this phenomenon was caused by lymphocyte redistribution but lymphocytes were not damaged. The mutated SIRPαD1-Fc molecule does not cause myelosuppression in bone marrow. Decrease of platelet and hemoglobin was transient and generally returned to baseline in 4 hours to 8 days. No treatment related hemolytic anemia and no permanent study discontinuation occurred.

SEQ ID NO: 1
EEELQVIQPDKSVSVAAGESAILHCTVTSLIPVGPIQWFRGAGPARELIY
NQKEGHFPRVTTVSESTKRENMDFSISISAITPADAGTYYCVKFRKGSPD
TEFKSGAGTELSVRAKPSAPVVSGPAARATPQHEPKSCDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
SEQ ID NO: 2
EEELQVIQPDKSVSVAAGESAILHCTVTSLIPVGPIQWFRGAGPARELIY
NQKEGHFPRVTTVSESTKRENMDFSISISAITPADAGTYYCVKFRKGSPD
TEFKSGAGTELSVRAKPSAPVVSGP
SEQ ID NO: 3
AARATPQHEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPGK

The clinical trial titled “Safety and Efficacy of IMM01 Plus Tislelizumab in Patients With Advanced Solid Tumors and Lymphomas” naming Zhengbo Song, Yan Zhang, Hui Zhu, Xu Tong, Shun Lu, and Yanqiu Zhao as Principal Investigators and Zhenzhen Zong, Xuan Zheng, Jiaqi Yang, and Rongqiang Sun as contacts (available at https://classic.clinicaltrials.gov/ct2/show/NCT05833984) is understood to have published on Apr. 27, 2023 on the NIH's ClinicalTrials.gov website, and it involves the use of IMM01, a mutated SIRPαD1-Fc molecule also known as timdarpacept, plus tislelizumab, in patients with advanced solid tumors and lymphomas (“Song”). While Song does not teach or suggest the inventions of this disclosure and can be read as teaching away from inventions of this disclosure, for purposes of United States Patent Law, Song constitutes a public disclosure of the herein-named inventor(s)' own information including that which is set forth in this disclosure. That is, we, named inventor(s) for purposes of United States Patent Law are affirmatively stating that the Apr. 27, 2023 Song publication is a publication of our work and information including that which is in this disclosure; and that the Song publication was authorized by us pursuant to our position with and obligation to assign to ImmuneOnco Biopharmaceuticals (Shanghai), Inc., the applicant of this disclosure (and hence Song is also a publication of applicant). We, named inventor(s), are, for purposes of United States Patent Law, affirmatively stating that Song is a disclosure by another (e.g., ImmuneOnco Biopharmaceuticals (Shanghai) Inc.) of our information (i.e., our subject matter), including that which is in this disclosure, and that Song is authorized by us in view of our position with and obligation to assign to ImmuneOnco Biopharmaceuticals (Shanghai), Inc., the applicant. We, named inventor(s), are also affirmatively stating that Song is hereby incorporated herein by reference, and that this disclosure is being filed within a year of the Apr. 27, 2023 disclosure of Song. Thus, we, named inventor(s) are affirmatively stating, for purposes of United States Patent Law, that it is our understanding that the Apr. 27, 2023 Song publication is disqualified from being prior art by the above statements concerning Song. And it is also our understanding that Song does not teach or suggest the inventions of this disclosure and can be read as teaching away from inventions of this disclosure.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

We claim:

1. A method of treating classic Hodgkin's lymphoma (cHL) in a subject in need thereof, comprising intravenously administering to the subject
i) a recombinant fusion protein at the dose of about 2.0 mg/kg body weight, once a week, and
ii) an anti-PD-1 antibody at the dose of about 200 mg, once every 3 weeks,
wherein the recombinant fusion protein comprises a mutated SIRPα D1 domain and a functional IgG1 heavy chain constant region, wherein the mutated SIRPα D1 domain comprises the amino acid sequence of SEQ ID NO: 2.

2. The method of claim 1, wherein the recombinant fusion protein comprises the amino acid sequence of SEQ ID NO: 1.

3. The method of claim 1, wherein the anti-PD-1 antibody is tislelizumab.

4. The method of claim 1, wherein when the recombinant fusion protein and the anti-PD-1 antibody are administered on the same day, the recombinant fusion protein is administered at least 30 minutes after completing the anti-PD-1 antibody administration.

5. The method of claim 1, wherein the recombinant fusion protein is administered via intravenous infusion, and the anti-PD-1 antibody is administered via intravenous infusion.

6. The method of claim 1, comprising the steps of
(a) administering intravenously to the subject about 200 mg of the anti-PD-1 antibody in the form of a composition comprising a pharmaceutically acceptable excipient and the anti-PD-1 antibody,
(b) administering intravenously to the subject about 2.0 mg/kg body weight of the recombinant fusion protein in the form of a composition comprising a pharmaceutically acceptable excipient and the recombinant fusion protein, and
(c) after steps (a) and (b), repeating step (a) once every 3 weeks, and repeating step (b) once weekly.

7. The method of claim 6, wherein Step (b) is performed at least 30 minutes after completing step (a).

8. The method of claim 6, wherein in step (c), when the recombinant fusion protein and the anti-PD-1 antibody are administered on the same day, the recombinant fusion protein is administered at least 30 minutes after completing the anti-PD-1 antibody administration.

9. The method of claim 1, wherein the method does not include administering a priming dose of the recombinant fusion protein or dose ramp-up administrations of the recombinant fusion protein to mitigate on-target anemia.

10. The method of claim 1, wherein the method i) produces more than 60%, 65%, 70%, 75%, 80%, 85%, 90% or 95% of objective response rate after 24 weeks or longer of the treating, wherein the objective response rate is defined as the sum of complete response (CR) rate and partial response (PR) rate, ii) produces more than 20%, 25%, 30%, 35%, 40%, or 45% of complete response rate after 24 weeks or longer of the treating, or iii) produces more than 85%, 90% or 95% of disease control rate after 24 weeks or longer of the treating, wherein the disease control rate is defined as the sum of complete response (CR) rate, partial response (PR) rate and stable disease (SD) rate.

11. The method of claim 1, wherein i) no more than 5% or 10% of treated subjects have chance of treatment-related hemolytic anemia, or ii) not more than 15% or 20% of treated subjects have chance of treatment discontinuation due to treatment-related adverse effects.

12. The method of claim 1, wherein the subject is with relapsed or refractory cHL.

13. The method of claim 12, wherein the subject experienced anti-PD-1 treatment failure.

14. A pharmaceutical composition comprising i) a recombinant fusion protein, and ii) an anti-PD-1 antibody, wherein the recombinant fusion protein comprises a mutated SIRPα D1 domain and a functional IgG1 heavy chain constant region, wherein the mutated SIRPα D1 domain comprises the amino acid sequence of SEQ ID NO: 2.

15. The pharmaceutical composition of claim 14, wherein the recombinant fusion protein comprises the amino acid sequence of SEQ ID NO: 1.

16. The pharmaceutical composition of claim 14, wherein the anti-PD-1 antibody is tislelizumab.
